(19) United States
(12) Patent Application Publication
(10) Pub. No.: US 2010/0048521 A1
(43) Pub. Date: Feb. 25, 2010

(54) COMBINATION OF A GLUCO-KORTICOID AND BETA-CYCLODEXTRIN CONJUGATED VITAMIN A-DERIVATE COMPLEX

(76) Inventors: Jan Wadstein, Malmo (SE); Erling Thom, Oslo (NO)

Correspondence Address: LADAS & PARRY LLP, 224 SOUTH MICHIGAN AVENUE, SUITE 1600, CHICAGO, IL, 60604, US

(21) Appl. No.: 12/441,947

(22) PCT Filed: Sep. 20, 2007
(86) PCT No.: PCT/NO07/00331

§ 371 Date: Jun. 25, 2009

(30) Foreign Application Priority Data
Sep. 20, 2006 (NO) 20064235

(51) Int. Cl. A61K 31/57; A61K 31/07; A61K 31/58; A61P 17/00

(52) U.S. Cl. 514/171; 514/552

(57) ABSTRACT

The present invention is related to a pharmaceutical combination of a glucocorticoid and a β-cyclodextrin conjugated vitamin A derivative complex as well as a pharmaceutical composition comprising the same. The combination and the composition of the invention can be used for reducing or eliminating skin atrophy induced by treatment with glucocorticoids (GC). Also comprised is a method for reducing or eliminating skin atrophy induced by GC treatment. Furthermore the invention concerns a kit comprising a glucocorticoid and a β-cyclodextrin conjugated vitamin A derivative complex.

### FIELD OF THE INVENTION

The present invention relates to a pharmaceutical combination of a glucocorticoid and a β-cyclodextrin conjugated vitamin A derivative complex, as well as a pharmaceutical composition comprising the same. The pharmaceutical combination and composition can be used for preventing, reducing or eliminating skin atrophy induced by treatment with glucocorticoids. The invention does also provide a method for preventing, reducing or eliminating skin atrophy induced by glucocorticoid treatment. Furthermore the invention concerns a kit comprising a glucocorticoid and a β-cyclodextrin conjugated vitamin A derivative complex.

### BACKGROUND OF THE INVENTION

The skin is the biggest organ of the human body and is very complex, due to its many functions. It should prevent the body from poisoning substances, which means it must have a strong barrier function. It should be able to sustain trauma of different kinds and if the skin is damaged it must be able to quickly repair itself.

Due to naturally occurring processes in the body the skin will age with time (chronological aging) and will eventually get wrinkled. However, the skin is also sensitive to various other factors such as, e.g., sun radiation, diseases, smoking, chemical exposure and radiation. Sun radiation causes aging of the skin. To much sun exposure will ultimately lead to cutaneous alterations such as wrinkling, leatheriness, loss of elasticity, looseness, roughness, hydration, etc. The cumulative effect of sunlight is referred to as photo-aging. Also medical treatment of the skin with steroids such as glucocorticoids will lead to degeneration.

Glucocorticoids (GCs), such as for example hydrocortisone, are highly effective for the topical treatment of inflammatory skin diseases and are a widely used class of anti-inflammatory drugs. Their long-term use, however, is accompanied by severe and partially irreversible adverse effects with atrophy being the most prominent. The cutaneous effects of GC-treatment are due to suppression of the proliferation and the extracellular matrix (ECM) protein synthesis of kerationcytes and fibroblast. The intercellular lipid layers are also reduced by GCs caused by the decreased synthesis of epidermal lipids, like ceramides, cholesterol and fatty acids. Thereby the stratum corneum gets thinner, followed by an increased transepidermal water loss. The skin loses its barrier function, its tensile strength and elasticity caused by the water loss and the degraded extracellular matrix. There is therefore a long felt medical need to alleviate and avoid these severe and unpleasant properties of the GCs felt by the patient during GC treatment.

The main objection of the present invention is thus to prevent, reduce or eliminate skin atrophy induced by GC treatment.

The clinical appearance of senile atrophy is similar to that of GC-induced skin atrophy (Schoeps S, Schäcke H, May E, Asadullah K, Glococorticoid therapy-induced skin atrophy, Exp Dermatol 2006; 15:406-420) and a number of studies have been performed and published showing that different A-vitamin derivatives have a significant effect on the aging of skin whether that is photo-aging, chronological aging or the type that is the most usual a combination of the two types.

A-vitamin acid (retinoic acid) it self has a well-documented effect on skin. In the cells A-vitamin acid has nutritional effects and promotes regeneration. However, this compound does also have side effects. Due to its irritant effect it can only be bought on prescription, and is used in certain skin diseases such as acne, psoriasis, eczema and others. Therefore, several vitamin A derivatives (retinyl derivatives) which are harmless to the skin have been used of which the following can be mentioned: retinol and ester of different types such as retinyl palmitate.

Also compositions comprising retinyl palmitate and hydrocortisone are known. US 2001/0006646 discloses formulations consisting essentially of insulin, hydrocortisone and a nutrient filled medium for stimulating wound healing of the skin. The nutrient filled medium includes among others vitamins such as for example retinyl palmitate. WO 96/07936 A2 discloses skin care compositions comprising an oil-in-water emulsion base containing retinoids selected from among others retinyl palmitate. Said skin care compositions may also contain a corticosteroid such as, for example, hydrocortisone.

The above mentioned compositions make use of retinyl palmitate, however, an essential condition for vitamin A preparations to have an effect is that they penetrate the skin in a satisfactory manner. Furthermore, it is necessary for them to be converted into vitamin A in the skin, since the cells have receptors for vitamin A acid it self only.

Vitamin A acid is relatively hydrophilic, whereas for instance the esters are relatively lipophilic. The conversion of vitamin A esters into vitamin A is effected by enzymatic splitting of the ester bond. Furthermore the skin must oxidize vitamin A to vitamin A acid. This conversion occurs in the deeper layers of the skin and not on the surface of the skin. Once hydrolysed to A-vitamin acid the acid can exert its beneficial effect without irritation.

To achieve a vitamin A ester preparation which is able to penetrate the skin the vitamin A ester can be conjugated to β-cyclodextrin. It has earlier been shown by Wadstein (1991) that vitamin A ester bound to β-cyclodextrin could penetrate the skin almost as effective as A-vitamin acid, but without the above mentioned side effects.

WO 94/21225 describes a skin care composition comprising as an active ingredient a conjugate of a vitamin A derivative and β-cyclodextrin, and pharmaceutically acceptable carriers and/or fillers. This application does also disclose the use of the above conjugate and, optionally colostrums for preparing a composition for the therapeutic or prophylactic treatment of aging symptoms in the skin. The vitamin A derivative is a retinyl ester, preferably retinyl palmitate.

Several studies with β-cyclodextrin conjugated retinyl palmitate (Thom E. Skin, “Treatment with two different galenical formulations of retinyl palmitate in humans”, J Appl Cosmetol 1993, 11, 71-76. Thom E., “Long-term effects after topical application of active retinyl palmitate”, J Appl Cosmetol 1994, 12, 25-30. Thom E. “A comparative double-blind within subject of the efficacy and tolerability of two different derivatives of Vitamin A on skin thickness and elasticity: Retinoic acid and conjugated retinyl palmitate”, J Appl Cosmetol, 1997, 15,133-138) have been performed.

This conjugate complex has two advantages. Firstly it will protect the vitamin A ester from oxidation and secondly it will increase the skin penetration and thus improve the amount of vitamin A in the skin where it is transformed to retinoic acid that can attach to the relevant receptors (Boehnlein J, Sakr A, Lichtin J L, Bronhaugh R L, “Characterization of sterase and alcohol dehydrogenase activity in, skin. Metabolism of retinyl palmitate to retinal (Vitamin A) during percutaneous absorption”, Pharmaceutical Research 1994; 8:1155115).

The clinical results from the vitamin A studies referred to above have been measured to be an increase in skin thickness and skin elasticity and an improved hydration capacity of the skin. These effects are probably due to an increase in the amount of elastin and fibril. The major skin collagen fibril, forming approximately 80% of the skin collagen, is type I collagen. The minor components are types III (10-15%), V (5%) and IV collagens (basement membrane).

In contrast to the well-investigated mechanism of type I collagen regulation, the mechanism for regulation of the type III collagen gene is far less understood. GC treatment reduces type III collagen with a more devastating effect than with type I collagen. This reflects the situation with fibrillar collagen: Type III collagen is more sensitive to GC treatment compared with type I collagen (Schoepe S, Schacke H, May E, Asadullah K. “Glucocorticoid therapy induced skin atrophy”, Exp Dermatol 2006; 15:406-420).

### SUMMARY OF THE INVENTION

Based on the above information it was surprising to discover that the present invention makes it possible to prevent, reduce or eliminate skin atrophy induced by GC treatment.

The present invention solves the problem by combining a glucocorticoid and a β-cyclodextrin conjugated vitamin A derivative complex.

Thus, in a first embodiment the present invention combines a glucocorticoid and a β-cyclodextrin conjugated vitamin A derivative complex and thus provides a pharmaceutical combination of a glucocorticoid and a β-cyclodextrin conjugated vitamin A derivative complex.

The active ingredients, the glucocorticoid and the β-cyclodextrin conjugated vitamin A derivative complex, can either be in the form a composition comprising both ingredients or can be in separate compositions to be used simultaneous or sequentially.

I one aspect the invention provides a pharmaceutical combination wherein said combination is a composition comprising a glucocorticoid and a β-cyclodextrin conjugated vitamin A derivative complex.

In another aspect the invention provides a pharmaceutical combination consisting of a composition comprising a glucocorticoid and a separate composition comprising a β-cyclodextrin vitamin A derivative complex. The compositions are to be used simultaneous or sequentially within a given time.

Furthermore, the present invention provides a pharmaceutical composition comprising a glucocorticoid and a β-cyclodextrin conjugated vitamin A derivative complex.

In another embodiment the invention concerns the combined use of a glucocorticoid and a β-cyclodextrin conjugated vitamin A derivative complex for preventing, reducing or eliminating skin atrophy induced by GC treatment.

In one aspect the invention concerns the use of a composition comprising a glucocorticoid and a β-cyclodextrin conjugated vitamin A derivative complex to prevent, reduce or eliminate skin atrophy induced by GC treatment.

In another aspect the invention concerns the use of a composition comprising a glucocorticoid and another composition comprising a β-cyclodextrin vitamin A derivative complex to prevent, reduce or eliminate skin atrophy induced by GC treatment. The compositions are to be used simultaneous or sequentially with in a given time.

In a further embodiment the invention concerns a method for reducing or eliminating skin atrophy induced by GC treatment in a patient.

In yet a further embodiment the invention concerns a kit comprising a glucocorticoid and a β-cyclodextrin vitamin A derivative complex.

### BRIEF DESCRIPTION OF THE DRAWINGS

FIG. 1:

Upper picture: Shows a cross-section of the skin at start (baseline).

Lower picture: Shows a cross-section of the skin after treatment with hydrocortisone for 4 weeks. The skin gets thinner and the density decrease.

FIG. 2:

Upper picture: Shows a cross-section of the skin at start (baseline).

Lower picture: Shows a cross-section of the skin after treatment with the combination of hydrocortisone and conjugated β-cyclodextrin for 4 weeks. The skin is almost unchanged with respect to thickness and density.

### DETAILED DESCRIPTION OF THE INVENTION

In a first embodiment the present invention makes it possible to prevent, reduce or eliminate skin atrophy induced by GC treatment by combining a glucocorticoid and a β-cyclodextrin conjugated vitamin A derivative complex, and thus provides a pharmaceutical combination of a glucocorticoid and a β-cyclodextrin conjugated vitamin A derivative complex. Also provide is a pharmaceutical composition comprising a glucocorticoid and a β-cyclodextrin conjugated vitamin A derivative complex. Vitamin A acid is also known as retinoic acid (3,7-dimethyl-9-(2,6,6-trimethyl-1-cyclohexen-1-yl)-2,4,6,8-nonatetraenoic acid). Retinoic acid is a physiological metabolite of retinol, occurring primarily as the all-trans form. Preferred vitamin A derivatives of the present invention are retinyl esters, especially retinyl palmitate.

The above mentioned vitamin A derivatives make complexes with β-cyclodextrin. Cyclodextrins are naturally occurring clathrates. They consist of homogeneous cyclic α-(1→4) linked D-glucopyranose units. When the number of α-D-glucopyranose is seven, the molecule is known as β-cyclodextrin or cycloheptaamylose. β-cyclodextrin has a hydrophobic cavity and form inclusion compounds with organic substances, such as, for example, vitamin A derivatives, salts and halogens in the solid state or in aqueous solutions.

The vitamin A derivative and the β-cyclodextrin are included in the conjugate in stoichiometric proportions, i.e., with a 1:2 ratio.

As used in the present invention, the term “glucocorticoid” means hormones of the arenal cortex having a stereoide character which especially affects the carbohydrate (sugar) and protein turnover, such as for example cortisol or hydrocortisone, cortisone, prednisone or prednisolone, desonid, metylprednisolone, prednicarbate, dexametasone, triamcinolone, betamethasone, deoximethasone, fluocinolonacetonid, fluocinoide, momethasone, fluticasone, prednyliden, metamethasone, clobetasol, etc. Glucocorticoids can be classified based on their potencies. Thus, class I GCs like hydrocortisone has weak effects regarding both desired anti-inflammatory effects and side effects, whereas class IV GCs like clobetasol propionate has strong effects. The glucocorticoid of the present invention is selected from classes I-IV GCs. Preferably the GCs are selected from hydrocortisone, triamcinolone, hydrocortisone butyrate, prednicarbarte, metylprednisolone, betamethasone, mometasone and clobetasol and the salts thereof, and most preferably the glucocorticoid is hydrocortisone.

Thus, in an embodiment the present invention combines a glucocorticoid and a β-cyclodextrin conjugated retinyl ester complex, such as β-cyclodextrin conjugated retinyl palmitate complex.

The active ingredients, the glucocorticoid and the β-cyclodextrin conjugated retinyl ester, such as β-cyclodextrin conjugated retinyl palmitate complex of the pharmaceutical combination, can either be in the form a composition comprising both ingredients or can be in separate compositions to be used simultaneous or sequentially.

In a further aspect the invention provides a composition comprising a glucocorticoid and a β-cyclodextrin conjugated retinyl ester complex, such as β-cyclodextrin conjugated retinyl palmitate complex.

In yet a further aspect the invention provides a pharmaceutical combination consisting of a composition comprising a glucocorticoid and a separate composition comprising a β-cyclodextrin retinyl ester complex, such as β-cyclodextrin conjugated retinyl palmitate complex. The compositions are to be used simultaneous or sequentially within a given time.

In the above mentioned compositions the glucocorticoid is selected from class I-IV GCs such as hydrocortisone, cortisone, prednisone or prednisolone, desonid, metylprednisolone, prednicarbate, dexametasone, triamcinolone, betamethasone, deoximethasone, fluocinolonacetonid, fluocinoide, momethasone, fluticasone, prednyliden, metamethasone, clobetasol and the salts thereof, and preferably the glucocorticoid is hydrocortisone.

Thus, in a preferred embodiment the invention provides a pharmaceutical combination combining hydrocortisone and a β-cyclodextrin conjugated retinyl palmitate complex.

In one aspect the invention provides a composition comprising hydrocortisone and β-cyclodextrin conjugated retinyl palmitate complex.

In another aspect the invention provides a combination consisting of one composition comprising hydrocortisone and a separate composition comprising β-cyclodextrin retinyl palmitate complex. The compositions are to be used simultaneous or sequentially within a given time.

The term “sequentially within a given time” means that the compositions can be applied to the skin simultaneous or within 30 minutes, preferably the compositions are applied separated by a time period of 10 minutes. The compositions can be applied in either order, e.g. the composition comprising the hydrocortisone can be applied first and then the composition comprising the β-cyclodextrin conjugated vitamin A derivative complex can be applied or vice versa. However, when the combination is used for prophylaxis, e.g. before radioactive treatment, the β-cyclodextrin conjugated vitamin A derivative complex will be applied first and the after an appropriate time period the GC will be applied. It is obvious that when a composition comprising both active components is used both components will be applied at the same time.

The amount of the β-cyclodextrin conjugated vitamin A derivative complex contained in the composition should be in the range of 0.1 wt % to 50 wt % of the total composition which corresponds to 100 to 50 000 IU of vitamin A.

In the compositions the glucocorticoid of grad 1, such as hydrocortisone, should be contained in an amount of 0.1 wt % to 10 wt % of the total composition. If a glucocorticoid of grade 3 is used, such as betmetasone, it should be contained in an amount of 0.05 wt % to 10 wt % of the total composition.

To make the compositions of the present invention acceptable to topical application they are mixed with ordinary pharmaceutically acceptable ingredients, adjuvantia, carriers and/or fillers commonly used in the art, for example anti-oxidants such as, e.g., tocopheryl acetate and tocopherol; conservation agents such as, e.g., methyl-p-hydroxybenzoate and acetyl-p-oxybenzoate; emulsifiers such as, e.g., Emulgator E 471 and E 472 c; fillers such as, e.g., olive oil, arachidis oleum, oleum soya, stearin, water; hydrogels such as triethanolamine, carboxy methyl cellulose, chitosan and resin.

Optionally lactose can be included in certain compositions of the present invention for treatments where it is important to promote the growth of the natural occurring acidofilic bacteria's as in eczema's or wounded skin. Lactose is a carbohydrate that exhibit different effects such as promotion of absorption of calcium and other minerals and promotion of growth of acidofilic bacteria's.

To regulate the pH of the compositions weak acids, normally used in compositions for application to the skin, can be used Also bicarbonate solutions such as sodium bicarbonate may be used to adjust the pH of the compositions. The pH of the compositions is adjusted to a pH between 2 and 8, preferably 5.5 which is an ideal acidity for active skincare.

The composition comprising a glucocorticoid and a β-cyclodextrin vitamin A derivative complex can be prepared by mixing the two components where the complex is added during the final step of preparation of the steroid composition. The temperature may vary between 6 to 70° C.

There are several different ways to prepare the β-cyclodextrin retinyl palmitate complex (see J. S. Pagington, Chemistry I Britain May 1987, Vol. 23). However, the present product is prepared by the following method: A solution of β-cyclodextrin, in an appropriate solvent such as, for example, water or an alcohol or mixtures thereof, is mixed with the inclusion compound, such as retinyl palmitate, solved in, e.g., isopropanol and stirred under cooling. After precipitation the complex is washed with water and solvent and then filtered of and dried. This inclusion compound is then used in the preparation of the final composition.

In another embodiment the invention concerns the use of a pharmaceutical combination of a glucocorticoid and a β-cyclodextrin conjugated vitamin A derivative complex, such as β-cyclodextrin conjugated retinyl palmitate complex to prevent, reduce or eliminate skin atrophy induced by GC treatment.

In one aspect the invention concerns the use of a pharmaceutical combination consisting of a composition comprising a glucocorticoid and a separate composition comprising a β-cyclodextrin retinyl ester complex, such as β-cyclodextrin conjugated retinyl palmitate complex to prevent, reduce or eliminate skin atrophy induced by GC treatment. The compositions are to be used simultaneous or sequentially with in a given time.

In another aspect the invention concerns the use of a composition comprising a glucocorticoid and a β-cyclodextrin conjugated vitamin A derivative complex, such as β-cyclodextrin conjugated retinyl palmitate complex to reduce or eliminate skin atrophy induced by GC treatment.

Thus, in a preferred embodiment the invention concerns the combined use of hydrocortisone and a β-cyclodextrin conjugated retinyl palmitate complex for reducing or eliminating skin atrophy induced by GC treatment. The combined use may either by provided by the use of one composition comprising both active ingredients or by two compositions each comprising one of the active ingredients. If the hydrocortisone and the β-cyclodextrin conjugated retinyl palmitate complex are contained in separate compositions they should be applied to the skin within a given time.

Furthermore, the invention concerns a method for reducing or eliminating skin atrophy induced by GC treatment comprising the application of a sufficient amount of a glucocorticoid and β-cyclodextrin derivate according to the invention on the skin of a human or animal subject.

Other indications for which this combination or composition can be beneficial are eczemas of different origin like atopical dermatitis, psoriasis and other conditions where the deeper layer of the skin is engaged preferably the collagen 1-3.

The use of this combination can also serve as a prophylactic treatment before radioactive treatment, e.g. in cancer, to protect the degeneration of the skin during such treatment. Furthermore the combination or product can be used before surgical treatment of thin skin to improve the final results of surgery. This is especially important in plastic surgery treatment.

The dosage will depend on the nature and severity of the condition(s) being treated, and possible associated treatments. The daily dosage of the compositions will range from 100 IU to 10 000 IU of vitamin A in one or more administrations, especially 1 to 3 administrations a day, depending on the condition being treated.

Also provided is a kit comprising a pharmaceutical combination or a pharmaceutical composition according to the present invention.

The following example illustrates the invention but does not limit it in any way.

Experimental Part

Comparative Study of Hydrocortisone and Hydrocortisone Combined with β-cyclodextrin Conjugated Retinyl Palmitate Complex with Respect to the Development of Skin Atrophy

The study was run according to the protocol described below and was approved by the local regional ethics committee before the study was initiated.

Protocol

The study included 10 healthy female volunteers. The average age was 43.2 years. They received treatment with hydrocortisone cream 1% on the inner part of one arm (right or left) once daily for 4 weeks. The area treated was marked and had an area of 10×15 cm. On the opposite arm they were treated with hydrocortisone cream 1% and after 10 minutes Nourella Cream containing β-cyclodextrin conjugated retinyl palmitate complex was applied (Pharma Medico; Aarhus, Denmark). Treatments were randomized so that half of the participants treated the right arm with hydrocortisone and the left with the combination and vice versa.

The skin was inspected clinically in the beginning and at the end of the study and objective measurement of the skin thickness and density was done by ultrasound (Dermascan C).

Results

The thickness of the skin (epidermis+dermis) was reduced in average by 15% on the arm treated with hydrocortisone indicating an atrophy effect, while the arm treated with the combination of hydrocortisone and β-cyclodextrin conjugated retinyl palmitate showed slight increase of 2% in thickness. It is a clear tendency in this material that the combination has anti-antrophogenic potential. However, due to the small sample investigated in this study the results are not reaching statistically significance (p=0.07). Results are shown in Table 1.

TABLE 1
Skin thickness in the two groups at baseline and after 4 week

| | Baseline (mm) | After 4 weeks (mm) |
|---|---|---|
| Hydrocortisone | 1.122 | 0.954 ns |
| Combination | 9.211 | 1.235 ns |

The evolution of dermis density is related to epidermis+dermis thickness. Low echogenic dark areas develop after treatment with GC while this development is not seen after treatment with the combination. This means that the anti-anthropogenic effect probably is due to sparing effect of the collagen. However, it is not a significant clinical effect as the material is too restricted, but it is a clear tendency (p=0.08). Results are shown in Table 2.

TABLE 2
Skin density in the two groups at baseline and after 4 week

| | Baseline | After 4 weeks |
|---|---|---|
| | (pixel) | (pixel) |
| Hydrocortisone | 1503.5 | 1830.0 ns |
| Combination | 1489.3 | 1499.6 ns |

Discussion

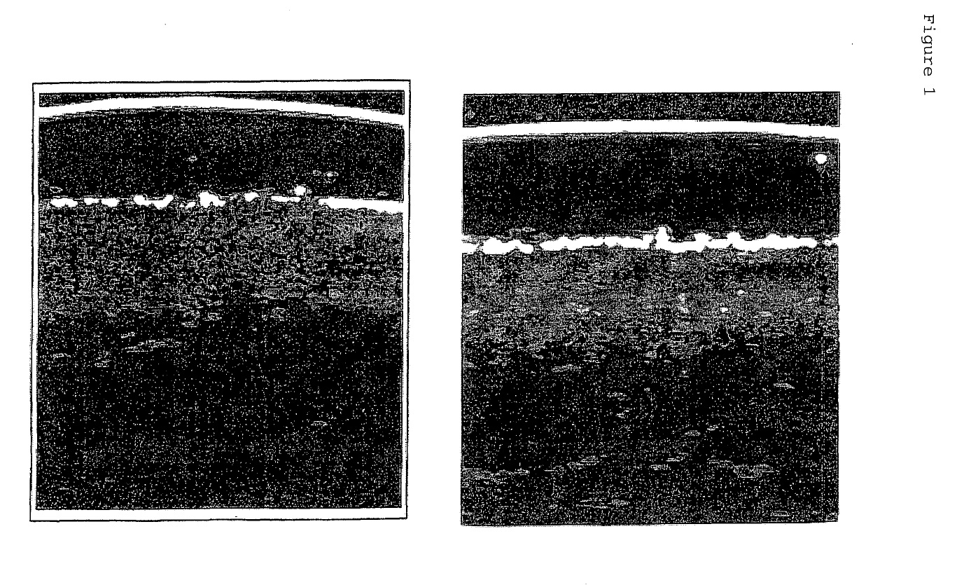

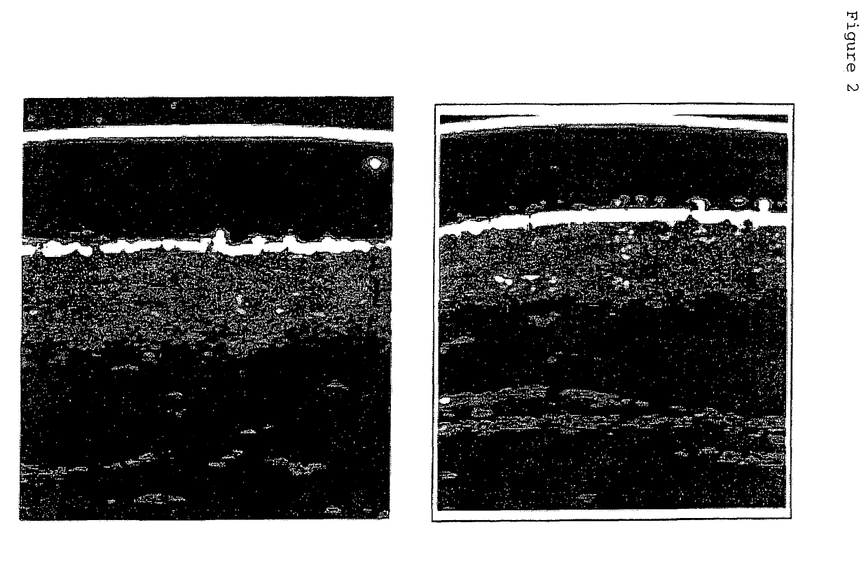

The results from the present study show, as has been shown by others, that continuous use of a GC class I (hydrocortisone) gives significant skin atrophy when used for 4 weeks (FIG. 1). This atrophy is normally reversible. When the hydrocortisone treatment is given as a combination treatment with β-cyclodextrin conjugated retinyl palmitate complex the skin atrophy is not detectable (FIG. 2). This combination is therefore an effective way to reduce and/or eliminate the most frequent side effect of GCs.

1. Pharmaceutical combination of a glucocorticoid and a β-cyclodextrin conjugated vitamin A retinyl palmitate complex.

2. Pharmaceutical combination according to claim 1, wherein said combination is a composition of a glucocorticoid and a β-cyclodextrin conjugated vitamin A retinyl palmitate complex.

3. Pharmaceutical combination according to claim 1, wherein said combination consists of a composition comprising a glucocorticoid and a separate composition comprising a β-cyclodextrin conjugated vitamin A retinyl palmitate complex.

4. Pharmaceutical combination according to claim 1, wherein the β-cyclodextrin and the vitamin A retinyl palmitate are included in the conjugate in stoichiometric proportions being a 1:2 ratio.

5. Pharmaceutical combination according to claim 1, wherein the glucocorticoid is selected from the group of classes I-IV glucocorticoids consisting of hydrocortisone, triamcinolone, hydrocortisone butyrate, prednicarbate, methylprednisolone, betamethasone, mometasone and clobetasol and the salts thereof.

6. Pharmaceutical combination according to claim 1, wherein the glucocorticoid is hydrocortisone.

7. Pharmaceutical composition comprising glucocorticoid and a β-cyclodextrin conjugated with vitamin A retinyl palmitate complex.

8. Pharmaceutical composition according to claim 7, wherein the β-cyclodextrin conjugated vitamin A retinyl palmitate complex is a β-cyclodextrin conjugated retinyl ester complex.

9. Pharmaceutical composition according to claim 7, wherein the β-cyclodextrin and the vitamin A retinyl palmitate included in the conjugate in stoichiometric proportions being a 1:2 ratio.

10. Pharmaceutical composition according to claim 7 wherein the glucocorticoid is selected from glucocorticoids of class I from the group consisting of hydrocortisone, triamcinolone, hydrocortisone butyrate, prednicarbate, methylprednisolone, betamethasone, mometasone and clobetasol and the salts thereof.

11. Pharmaceutical composition according to claim 7 wherein the glucocorticoid is hydrocortisone.

12. Pharmaceutical composition according to claim 7, wherein the amount of the β-cyclodextrin conjugated vitamin A retinyl palmitate complex contained in the composition is in the range of 0.1 wt % to 50 wt % and the amount of glucocorticoid contained in the composition is in the range of 0.05 wt % to 10 wt % of the total composition.

13. Pharmaceutical composition according to claim 7 wherein said composition further comprises at least one of pharmaceutically acceptable ingredients, adjuvantia, carriers, and fillers.

14. (canceled)

15. (canceled)

16. (canceled)

17. A method for reducing or eliminating skin atrophy induced by GC treatment comprising the step of applying a pharmaceutical combination according to claim 1 on the skin of a human or animal subject.

18. A Kit comprising a glucocorticoid and a β-cyclodextrin conjugated vitamin A retinyl palmitate complex.

19. Kit according to claim 18, wherein the kit comprises a pharmaceutical combination according to claim 1 or a pharmaceutical composition according to claim 7.

20. A method for reducing or eliminating skin atrophy induced by GC treatment comprising the step of applying a pharmaceutical composition according to claim 7 on the skin of a human or animal subject.

21. The method of claim 17 wherein the combination is a composition of a glucocorticoid and a separate β-cyclodextrin vitamin A retinyl palmitate complex composition and each composition is used simultaneously or sequentially within a given time.
